(19) United States
(12) Patent Application Publication
(10) Pub. No.: US 2024/0238333 A1
(43) Pub. Date: Jul. 18, 2024

(54) TREATMENT OF DELIRIUM USING NITROUS OXIDE

(71) Applicant: Penland Foundation, Beaumont, TX (US)

(72) Inventor: Roland M. Williams, Beaumont, TX (US)

(21) Appl. No.: 18/392,640

(22) Filed: Dec. 21, 2023

Related U.S. Application Data
(60) Provisional application No. 63/479,700, filed on Jan. 12, 2023.

(51) Int. Cl. A61K 33/00; A61K 9/00; A61K 31/519; A61K 31/714; A61P 25/00

(52) U.S. Cl. CPC A61K 33/00; A61K 9/0043; A61K 31/519; A61K 31/714; A61P 25/00

(57) ABSTRACT

Provided is a method of treating delirium in a patient in need thereof, the method comprising: administering nitrous oxide and oxygen to the patient and B-12 and/or folic acid, thereby treating delirium. A composition of the nitrous oxide and oxygen may comprise from about 1% nitrous oxide/about 99% oxygen to about 70% nitrous oxide/about 30% oxygen. The method may further comprise monitoring if the patient's neuroexcitatory substances level normalizes.

### CROSS-REFERENCE TO RELATED APPLICATION

This application claims the benefit of U.S. Provisional Application No. 63/479,700, filed Jan. 12, 2023, which is incorporated by reference herein in its entirety.

### BACKGROUND

### Technical Field

The present disclosure generally relates to a theory of the mechanism of the development of delirium—the pathological activations of the cytokine system, which results in excess substance P which causes immune cells to chronically release cytokines. It also results in excess glutamate production which causes neuron death by neuron excitatory toxicity. The present disclosure also relates to methods for treating, alleviating, and/or preventing this neurological disruption of the normal levels of neurotransmitters in the brain, which causes cerebral dysfunction and results in an acute confusional state. Triggering conditions include but not limited to prolonged sleep deprivation, environmental, physical/psychological stress, infections, medications, primary neurological diseases, concurrent illness, surgery, renal conditions, and traumatic brain injury which can cause an elevation of substance P levels. This results in elevated serum and brain cytokine levels.

### Background

Cytokine production is a normal part of infection control and damage repair. However, if overproduced, acutely or chronically, this can affect sensory, neurotransmitter, neuroendocrine, or neuroinflammatory pathways. Substance P activates the NK-1-3 receptors on immune cells, triggering the release of cytokines. Nitrous oxide by one of its mechanisms of action can mitigate the overproduction of acute levels of substance P. This can suppress and control the elevated cytokine levels that can affect normal neurotransmitter levels in the brain.

### SUMMARY

The present disclosure is related to a method of treating delirium in a patient in need thereof, the method comprising: administering nitrous oxide and oxygen to the patient by inhalation, thereby treating delirium. A composition of the nitrous oxide and oxygen may comprise from about 1% nitrous oxide/about 99% oxygen to about 70% nitrous oxide/about 30% oxygen. Preferably, the composition of the nitrous oxide and oxygen may comprise from about 40% nitrous oxide/about 60% oxygen to about 50% nitrous oxide/about 50% oxygen. More preferably, the composition of the nitrous oxide and oxygen may comprise about 50% nitrous oxide/about 50% oxygen.

The nitrous oxide and oxygen may be provided to an adult who weighs about 150 lbs. between about for about between 1 minute and about 1 hour every about 4-8 hours. Preferably, the nitrous oxide and oxygen may be provided to an adult who weighs about 150 lbs. between about for about 20 minutes every about 6-8 hours. A composition, duration, interval, and total amount of the inhaled anesthetics provided to an adult, or a child may be adjusted for age, weight, or a combination thereof. The nitrous oxide and oxygen may be provided by continuous administration over the period of time.

The method may further comprise monitoring if the patient's neuroexcitatory substances level normalizes. The neuroexcitatory substances may comprise substance P or glutamate. The monitoring if the patient's neuroexcitatory substances level normalizes may comprise monitoring if the patient's blood glutamate and a substance P level normalizes. The patient's neuroexcitatory substances level may be the blood level. Preferably, a normal blood glutamate level may be from about 40 to about 60 uM.

If the patient's neuroexcitatory substances level fails to normalize, additional nitrous oxide and oxygen may be administered to the patient by inhalation.

The method may further comprise monitoring the patient's symptoms. If the patient's physical symptom continues, additional nitrous oxide and oxygen may be administered to the patient.

The method may further comprise monitoring the patient's mental symptoms and/or blood tests to measure inflammatory mediators such as, C-reactive protein or sedimentation rates.

The method may further comprise administering B-12 and/or folic acid (e.g., 300 mcg folic acid sublingual or by injection and/or B-12 1,000 mcg by mouth daily) to the patient.

### DETAILED DESCRIPTION OF THE PREFERRED EMBODIMENTS

Further in relation to this, before explaining at least the preferred embodiments of the disclosure in greater detail, it is to be understood that the disclosure is not limited in its application to the details of construction and to the arrangements of the components set forth in the following description. It would be understood by those of ordinary skill in the art that embodiments beyond those described herein are contemplated, and the embodiments can be practiced and carried out in a plurality of different ways. Also, it is to be understood that the terminology used herein is for the purpose of description and should not be regarded as a limiting factor.

Unless otherwise defined, the terms used herein refer to that which the ordinary artisan would understand such term to mean based on the contextual use of such term herein. To the extent that the meaning of a term used herein as understood by the ordinary artisan based on the contextual use of such term differs in any way from any dictionary definition of such term, it is intended that the meaning of the term as understood by the ordinary artisan will prevail.

As used herein, the term “about” means approximately or nearly and in the context of a numerical value or range set forth herein means ±10% of the numerical value or range recited or claimed.

The term “treating” includes delaying, alleviating, mitigating, or reducing the intensity, progression, or worsening of one or more attendant symptoms of a disorder or condition and/or alleviating, mitigating, or impeding one or more causes of a disorder or condition. Treatment under the claimed disclosure may be a preventative treatment, prophylactic treatment, remission of treating, or ameliorating treatment.

The term “therapeutically effective amount” or “therapeutically effective dose” refers to the amount of a composition, compound, therapy, or course of treatment that, when administered to an individual for treating a disorder or disease, is sufficient to affect such treatment for the disorder or disease. The “therapeutically effective amount” will vary depending on the composition, the compound, the therapy, the course of treatment, the disorder or disease and its severity, and the age, weight, etc., of the individual to be treated.

As used herein, “consists essentially of” when used in conjunction with a composition means excluding other materials that contribute to mitigating cytokine overproduction, thereby treating delirium that has resulted from the overproduction of cytokines. The objective of administering inhaled anesthetic by inhalation is to treat the conditions by mitigating cytokine overproduction. With the language, other materials that contribute to the treatment that materially affect the basic and novel characteristics of the disclosure are not required and are potentially counterproductive because they may offset the treatment effect of inhaled anesthetic. In other words, the meaning of “consists essentially of” is tied to the objective and excludes materials (that contribute to the treatment) that are pharmaceutically active for the treatment and materially mitigate cytokine overproduction and thereby affecting the treatment of the conditions. Small traces that have little or no effect to the treatment as part of the embodiments of the presentation disclosure may exist in a composition that consists essentially of inhaled anesthetic under the definition because it would not materially affect its function and/or objective.

In accordance with the principles of the present disclosure, use of nitrous oxide is recommended to treat delirium that results in excess activation of the cytokine system (cytokine storm).

### Delirium

### (Acute Confusional State; Toxic or Metabolic Encephalopathy)

### Definition

Delirium is an organically caused decline from a previous baseline mental functioning and develops over a short period of time, typically hours to days.
Delirium is a syndrome encompassing disturbances in attention, consciousness, and cognition.
Delirium may also involve other neurological deficits, such as psychomotor disturbances (e.g., hyperactive, hypoactive, or mixed), impaired sleep-wake cycle, emotional disturbances, and perceptual disturbances (e.g., hallucinations and delusions), although these features are not required for diagnosis.
Delirium exists as a stage of consciousness somewhere in the spectrum between normal awakeness/alertness and coma.

### Clinical Features/Signs and Symptoms:

Inattention
Memory impairment
Disorientation
Clouding of consciousness
Disorganized thinking/thought disorder
Diffuse cognitive impairment
Delusions
Hallucinations/perceptual change
Language disturbances
Sleep changes
Psychotic
Mood lability
Motor activity changes
   Hyperactive
   Hypoactive

### Causes:

Delirium arises through the interaction of a number of predisposing and precipitating factors.
Individuals with multiple and/or significant predisposing factors are highly at risk for suffering an episode of delirium with a single and/or mild precipitating factor. Conversely, delirium may only result in healthy individuals if they suffer serious or multiple precipitating factors. It is important to note that the factors affecting those of an individual can change over time, thus an individual's risk of delirium is dynamic.
   Predisposing Factors
      65 or more years of age
      Male sex
      Cognitive impairment/dementia
      Physical comorbidity (biventricular failure, cancer, cerebrovascular disease
      Psychiatric comorbidity
      Sensory impairment (vision, hearing)
      Functional dependence (e.g., requiring assistance for self-care or mobility)
      Dehydration/malnutrition
      Drugs and drug-dependence
      Alcohol dependence
   Precipitating Factors
      Acute confusional state may be caused by alcohol withdrawal, also known as delirium tremens.
      Any acute factors that affect neurotransmitter, neuroendocrine, or neuroinflammatory pathways can precipitate an episode of delirium in a vulnerable brain.
      Clinical environments can also precipitate delirium.
      Some of the most common precipitating factors are listed below:
         Prolonged sleep deprivation
         Environmental, physical/psychological stress
         Inadequately controlled pain
         Admission to an intensive care unit
         Immobilization, use of physical restraints
         Urinary retention, use of bladder catheter
         Emotional stress
         Severe constipation/fecal impaction
         Medications
         Sedatives
         Benzodiazepines
         Opioids
         Anticholinergics
         Dopaminergic
         Corticosteroids
         Polypharmacy
         General anesthetic
         Substance intoxication or withdrawal
         Primary neurologic diseases
         Severe drop in blood pressure, relative to the patient's normal blood pressure (orthostatic hypotension) resulting in inadequate blood flow to the brain (cerebral hypoperfusion)
         Stroke/Transient ischemic attack (TIA)
         Intracranial bleeding
         Meningitis, encephalitis
         Concurrent illness
         Infections—especially respiratory (pneumonia, COVID-19) and urinary tract infections
         Iatrogenic complications
         Hypoxia, hypercapnia, anemia
         Poor nutritional status, dehydration, electrolyte imbalances, hypoglycemia
         Shock, heart attacks, heart failure
         Metabolic derangements (SIADH, Addison's disease, hyperthyroidism)
         Chronic/terminal illness (cancer)
         Post-traumatic event (fall, fracture)
         Mercury poisoning (Erethism)
         Surgery
         Cardiac, orthopedic, prolonged cardiopulmonary bypass, thoracic surgeries

### Pathophysiology:

Mechanisms are not fully understood but may involve:
   Reversible impairment of cerebral oxidative metabolism
   Multiple neurotransmitter abnormalities, especially cholinergic deficiency
   Generation of inflammatory markers, including C-reactive protein, interleukin-1 beta and 6, and tumor necrosis factor-alpha
Stress of any kind upregulates sympathetic tone and downregulates parasympathetic tone, impairing cholinergic function and thus contributing to delirium. Older people are particularly vulnerable to reduced cholinergic transmission, increasing their risk of delirium.
Regardless of the cause, the cerebral hemispheres or arousal mechanisms of the thalamus and brain stem reticular activating system become impaired.

### Hypothesis:

Hypotheses about the pathophysiology of delirium are speculative and largely based on animal research.
According to the neurotransmitter hypothesis, decreased oxidative metabolism in the brain causes cerebral dysfunction due to abnormalities of various neurotransmitter systems.
Reduced cholinergic function, excess release of dopamine, norepinephrine, and glutamate, and both decreased and increased serotonergic and gamma-aminobutyric acid activity may underlie the different symptoms and clinical presentations of delirium.
According to the inflammatory hypothesis, increased cerebral secretion of cytokines due to a wide range of physically stressful events plays an important role in the occurrence of delirium. Since cytokines can influence the activity of various neurotransmitter systems, these mechanisms may interact. Also, more fundamental processes like intraneuronal signal transduction, second messenger systems that at the same time use neurotransmitters as first messengers and play an important role in their synthesis and release, may be disturbed.
Severe illness and physiologic stress may give rise to modification of blood-brain barrier permeability, the sick euthyroid syndrome with abnormalities of thyroid hormone concentrations, and increased activity of the hypothalamic-pituitary-adrenal axis. These circumstances possibly also contribute to changes in neurotransmitter synthesis and release of cytokines in the brain, and consequently to the occurrence of delirium.
Elderly patients are more at risk for developing delirium, very likely due to age-related cerebral changes in stress-regulating neurotransmitter and intracellular signal transduction systems.

### Diagnosis:

Mental status examination
Standard diagnostic criteria to confirm delirium
Thorough history
Directed physical examination and selective testing to determine cause

### Prognosis:

Morbidity and mortality rates are high in patients who have delirium and are admitted to the hospital or who develop delirium during hospitalization.
   35 to 40% of hospitalized patients with delirium die within 1 year.
   These rates may be high partly because such patients tend to be older and to have other serious disorders.
Delirium due to certain conditions (e.g., hypoglycemia, drug or alcohol intoxication, infection, iatrogenic factors, drug toxicity, electrolyte imbalance) typically resolves rapidly with treatment.
Recovery may be slow (days to even weeks or months), especially in older patients, resulting in longer hospital stays, increased risk and severity of complications, increased costs, and long-term disability. Some patients never fully recover from delirium.
For up to 2 years after delirium occurs, risk of cognitive and functional decline, institutionalization, and death is increased.
In a recent meta-analysis, delirium in surgical and nonsurgical patients was significantly associated with cognitive impairment lasting ≥3 months after the delirium episode.

### Treatment:

Correction of the cause and removal of aggravating factors
   Correcting the cause (e.g., treating infection, giving fluids and electrolytes for dehydration) and removing aggravating factors (e.g., stopping drugs) may result in resolution of delirium. Nutritional deficiencies of, e.g., thiamin or vitamin B12 should be corrected, and good nutrition and hydration should be provided.
Supportive care
   The environment should be stable, quiet, and well-lit and include visual cues to orient the patient (calendar, clocks, family photographs).
   Frequent reorientation and reassurance by hospital staff or family members may also help.
   Sensory deficits should be minimized (by replacing hearing-aid batteries, by encouraging patients who need eyeglasses or hearing aids to use them).
   Approach to treatment should be interdisciplinary (with a physician, physical and occupational therapists, nurses, and social workers); it should involve strategies to enhance mobility and range of motion, treat pain and discomfort, prevent skin breakdown, ameliorate incontinence, and minimize risk of aspiration.
Management of agitation
   Agitation may threaten the well-being of the patient, a caregiver, or a staff member. Simplifying drug regimens and avoiding use of IV lines, bladder catheters, and physical restraints (particularly in the long-term care setting) as much as possible can help prevent exacerbation of agitation and reduce risk of injury.
   In certain circumstances, physical restraints may be needed to prevent patients from harming themselves or others. Restraints should be applied by a staff member trained in their use; they should be released at least every 2 hours to prevent injury and discontinued as soon as possible. Use of hospital-employed assistants (sitters) as constant observers may help avoid the need for restraints.
   Explaining the nature of delirium to family members can help them cope. They should be told that delirium is usually reversible but that cognitive deficits often take weeks or months to abate after resolution of the acute illness
Drugs
   Drugs, typically low-dose haloperidol (0.5 to 1.0 mg orally, IV, or IM once, then repeated every 1 to 2 hours as needed)
      Drugs may lessen agitation or psychotic symptoms; occasionally, much higher doses are necessary.
      However, drugs do not correct the underlying problem and may prolong or exacerbate delirium.
   Second-generation (atypical) antipsychotics
      They have fewer extrapyramidal adverse effects.
      Long-term use may cause weight gain and hyperlipidemia and increase the risk of type 2 diabetes.
      In older patients with dementia-related psychosis, these drugs increase risk of stroke and death. These drugs are not typically given IV or IM.
         risperidone 0.5 to 3 mg orally every 12 hours
         olanzapine 2.5 to 15 mg orally once a day
         quetiapine 25 to 200 mg orally every 12 hours
      Benzodiazepines
         Benzodiazepines are the drugs of choice for delirium caused by withdrawal from alcohol. Their onset of action is more rapid (5 minutes after parenteral administration) than antipsychotics.
         Benzodiazepines should be avoided if delirium results from other conditions because these drugs worsen confusion and sedation.
      lorazepam 0.5 to 1.0 mg orally or IV once, then repeated every 1 to 2 hours as needed.

### The Role of Inflammation in the Pathogenesis of Delirium in Older Adults

Inflammatory markers have been shown to be elevated in delirium, thereby implicating inflammation as a possible mediating factor in its genesis. There is evidence in both basic science and clinical research literature that elevated cytokines play a crucial role in the development of cognitive dysfunction observed in delirium.

### Introduction

The human immune system is a complex system of adaptive and innate responses that provides protection from external and internal threats. The cells of the immune system (e.g., neutrophils and macrophages) communicate via cytokines, which are hormone-like proteins produced by a variety of immune cells. Cytokines are important mediators of the immune response, which initiate, perpetuate, or downregulate the response. Cytokines include interleukins (IL), tumor necrosis factors (TNF), and transforming growth factors (TGF-ß 1-3). Certain cytokines are considered proinflammatory (IL-1, IL-6, and TNF-α), and others are anti-inflammatory (IL-4, IL-10, and IL-13).

### Inflammation and Cognition

Delirium is an acute decline in cognition, and is associated with acute inflammatory states. Studies examining the relationship of acute inflammation on cognitive decline in patients with delirium showed that acute systemic inflammatory events were associated with an increase in serum levels of TNF-α and a 2-fold increase in rate of cognitive decline over a 6-month period. Subjects with higher baseline levels of TNF-α were more likely to have delirium and were associated with a 4-fold increased rate of cognitive decline over a 6-month period compared to those with low TNF-α levels.

### Systemic Inflammation and the Brain

Despite the protection of the blood-brain barrier, it is now recognized that the brain is in communication with the immune system, thereby allowing systemic, peripheral inflammatory reactions to influence brain function, making the brain susceptible to the consequences of systemic inflammation. It appears that peripheral cytokines can influence cognition directly through effects on neurotransmission, sleep, and nutritional intake. One proposed mechanism by which peripheral cytokines influence the brain is through cellular communication across the blood-brain barrier. Studies show that systemic inflammation activates vascular endothelial cells and perivascular cells located at the blood-brain barrier, which propagates the inflammatory cascade and directly or indirectly injures neurons. A second mechanism by which cytokines may influence the brain is through stimulation of peripheral sensory neural affects, such as the Vagus nerve, which leads to central cytokine production. A third mechanism by which cytokines influence the brain is through altered neurotransmission, and in support of this, it has been shown that both central and peripheral cytokines alter noradrenergic, dopaminergic, and serotonergic metabolism in the brain.

### Inflammatory Markers in Delirium

Dysregulation of cytokines is believed to be the key inciter of neurodegeneration and subsequent cognitive impairment in delirium that results from activation of the systemic inflammatory cascade. Therapeutic and investigational uses of interleukins in human and rat models induce symptoms of delirium, alter acetylcholine levels and activity, and mediate exotoxic neurodegeneration. Risk factors for cytokine dysregulation include age as well as acute illness, infection, and trauma. Aging alters CNS and peripheral levels of certain cytokines even in the absence of disease or inflammation. Further, studies in mice indicate that aging brains are predisposed to exacerbated neuroinflammatory cytokine responses as a result of changes in glial reactivity. It is also becoming clear that the impact of peripheral inflammatory signals on the brain depends on existing levels of inflammation, as occurs in dementia or other direct brain lesions. Administration of therapeutic levels of systemic cytokines in oncology patients can result in neuropsychiatric side effects, including impaired thought processing. Peripheral cytokines (along with prostaglandins) are also thought to be responsible for the central behavioral changes seen with the syndrome of “sickness behavior,” which refers to a constellation of symptoms associated with illness and delirium, such as lethargy, decreased learning, reduced mobility, reduced social activity, and increased sleep. Sickness behavior is seen in conditions associated with systemic inflammation, such as infection or acute flares of chronic disease.

Cytokine dysregulation can lead to neuronal injury through a variety of mechanisms, including (1) altered neurotransmission, (2) apoptosis, and (3) activation of microglia and astrocytes which leads to production of free radicals, complement factors, glutamate, and nitric oxide. Also, the consequences of “sickness behavior” induced by cytokine dysregulation, including altered sleep, anorexia, and impaired nutrition may further impair cognition. Activation of the inflammatory cascade alters peripheral and/or central cytokines, leading to a central neurodegenerative inflammatory process that becomes clinically apparent as cognitive impairment when the cognitive reserve is breached.

### Clinical Studies of Inflammation and Delirium

To further understand the role that inflammation plays in delirium, researchers have studied and measured levels of inflammatory markers in acutely ill patients. Precipitants of delirium, such as extracerebral infection, general surgery, head trauma, and cerebral ischemia, induce systemic or local acute phase responses, often in association with delirium. After cardiac surgery, for example, elevated levels of chemokines in a case-control trial were associated with delirium, although methods of measurement did not allow analyses of the roles of individual cytokines.

### CONCLUSION

As people age, the regulatory mechanism of brain and whole body suffers loss of efficiency and adaptability to sudden internal and external trauma. No matter what the trauma is, the result is an increase in glutamate and substance P in the brain and plasma. The elevation in substance P results in activation of immune cells to produce cytokines peripherally and centrally. The aging brain struggles to adapt to these biochemical changes and neurotransmitter imbalances. This causes the varying central, peripheral, motor, and cognitive symptoms of delirium. The site in the brain most affected, levels above normal levels of glutamate and substance P, and duration of these elevation cause the spectrum, of symptoms that comprise delirium.

### Treatment of Delirium

Nitrous oxide has unique properties besides central anxiety control or sedation. Studies in nitrous oxide have shown that it has the ability to suppress the tachykinins, substance P and glutamate in the dorsal root, trigeminal and vagus sensory ganglia. Clinical observation has shown that this suppression lasts about 6-8 hours with the use of conscious sedation levels (e.g., about 50% nitrous oxide/about 50% oxygen. Substance P triggers immune cells to produce cytokines. Glutamate increases excitatory stimulation of the brain. Since the half-life of Substance P, glutamate and the cytokines is seconds to minutes, the effect should be immediate.

In an embodiment, if a patient suffers a delirium event, then the treatment is about 50% nitrous oxide/about 50% oxygen for 30 minutes, every 6-8 hours. The nitrous oxide to oxygen ratio, duration of dose, and time between dosages could be titrated to individual patient needs. Symptoms of elevated serum glutamate and substance P levels can be evaluated to determine length of treatment. Length of treatment may be influenced by the precipitating factor and the resolution of the factor that initiated the delirium event.

Nitrous oxide does not cause respiratory depression, over sedation, or irritation of the lungs at effective dosages with little or no side effects. The inhaled dosage can be from about 1% nitrous oxide/about 99% oxygen to about 70% nitrous oxide/about 30% oxygen depending on individual needs and sensitivity. Clinical indications suggest that about 40% nitrous oxide/about 60% oxygen to about 50% nitrous oxide/about 50% oxygen would be optimal.

Time of inhalation would vary from one minute to one hour with 20 minutes being the optimal time frame. A longer time may be used if necessary. For example, the nitrous oxide and oxygen may be administered to an adult who weighs about 150 lbs. for about between 1 minute and about 1 hour every about 4-8 hours; or for about 20 minutes every about 6-8 hours. The nitrous oxide and oxygen may be administered by continuous administration over the period of time.

Duration of substance P suppression can be from 1 minute to 12 hours with average cases of 4-8 hours of substance P suppression. The length of treatment to suppress the severe cytokine reaction will vary and be influenced by the time to medically correct the initiating factor. The nitrous oxide mechanism is suppression of substance P in the peripheral spinal and vagus ganglia. The amount of nitrous oxide or inhaled anesthetics used, duration of administration, and length of effectiveness will have to be titrated to the individual. For children, adjustments will have to be made for age and body weight. Other inhaled anesthetics may have to be used at a different oxygen % than the nitrous oxide to produce effective clinical results. Other inhalants can be included in the above-compositions such as for other purposes.

If side effects from too much substance P suppression nitrous oxide use occur, then inhalation may be reduced or eliminated. Before, during, and after the provision of the inhaled nitrous oxide and oxygen, blood tests may be done to monitor and assess the patient's cytokine level including a substance P level and a viral load. Blood glutamate levels could be monitored to make sure that levels drop to normal, and physical symptoms monitored to make sure they normalize as well (developmental milestones charted). Normal blood glutamate levels may range from 40 to 60 uM. Alternatively, normal blood glutamate levels may be one a person skilled in the art would reasonably perceive. In addition, before, during and after the inhalation of nitrous oxide and oxygen, a blood oxygen level and a pulse may be monitored and assessed. When blood tests show an increase in substance P, glutamate, or CGRP and/or the symptoms begins to re-develop, more nitrous oxide/oxygen can be given to combat this effect.

If nitrous oxide treatments must be extended over one day, then B-12 and/or folate (folic acid) vitamin supplements (e.g., 300 mcg folic acid sublingual or by injection and/or B-12 1,000 mcg by mouth daily) may need to be administered as long term Nitrous Oxide use can result in depletion of the important vitamins.

As mentioned, long term nitrous oxide treatment may have toxic side effects, and folic acid and/or B-12 should be administered. The folic acid and/or B-12 can be administered prior to beginning the treatment period (multiple treatments over time) and/or during or after the treatment period. The physician can require that the patient take the folic acid and/or B-12 and in some case, can conduct tests to determine compliance. Compliance can be performed in other ways such as by the physician asking questions or other step (or combination of steps).

In some embodiments, a composition administered to a patient consists of nitrous oxide and oxygen.

In some embodiments, a composition administered to a patient consists essentially of nitrous oxide and oxygen.

In some embodiments, a pharmaceutically active composition contained in a composition administered to a patient consists of nitrous oxide and oxygen.

The composition may additionally include a pharmaceutically inactive composition.

### Prevention of Delirium

If a patient has predisposing factors and suffers precipitating conditions, then, said patient could be treated with nitrous oxide to prevent the surge in substance P and glutamate levels with the resulting central neuroexcitation and cytokine-induced inflammation. The treatment would be 50% nitrous oxide/50% oxygen for 30 minutes every 6 hours until the precipitating event has been resolved. Serum glutamate and substance P levels can be monitored to determine when levels have returned to normal. For example, if a patient has had previous delirium event or has predisposing factors and undergoes surgery. Then the patient could be treated with nitrous oxide, post surgery, to prevent elevated cytokine levels and mitigate or prevent delirium event.

The invention is further described in the following prophetic examples. These examples are for illustrative purposes only, and are not to be construed as limiting the appended claims.

### Example 1

A 68 year old male patient suffers from delirium. The patient weighs about 170 lbs. The patient primarily has an issue with inattention, memory impairment, and delusions. He receives 50% nitrous oxide/50% oxygen by inhalation for appointments 30 minutes each visit for a week. Within minutes of the administration of the nitrous oxide, he is completely lucid. He is capable of recalling many events he couldn't recall before the administration. His focus level significantly improves after the administration. The patient and his family reports significant improvement in the brain function without administering medications such as haloperidol. After the administration, substance P and glutamate blood levels are measured to be lower than before. The patient optionally receives additional treatments such as non-pharmacologic treatment and active exercise. The patient also receives B-12 and/or folate vitamin supplements.

### Example 2

A 78 year old female patient suffers from delirium with alcohol dependence. The patient weighs about 145 lbs. The patient primarily has an issue with inattention, disorientation, hallucinations, and language disturbances. She receives 50% nitrous oxide/50% oxygen by inhalation for appointments every 6 hours for one day. Within minutes of the administration of the nitrous oxide, she is completely lucid. She is capable of having proper conversation. Her cognition level significantly improves after the administration. The patient and her family reports significant improvement in the brain function without administering medications such as benzodiazepines. After the administration, substance P and glutamate blood levels are measured to be lower than before. The patient optionally receives additional treatments such as non-pharmacologic treatment and active exercise.

### Example 3

A 72 year old male patient suffers from cognitive impairment with drug dependence. The patient weighs about 171 lbs. The patient is withdrawn from an intensive care unit. He suffers from sensory impairment and requires assistance for self-care. While he is not currently diagnosed with delirium, his family wants to prevent him from developing any potential delirium. He receives 50% nitrous oxide/50% oxygen by inhalation for appointments every 8 hours for two weeks. Within minutes of the administration of the nitrous oxide, he is completely lucid. She is capable of having proper conversation. His cognition level significantly improves after the administration. The patient and his family reports significant improvement in the brain function without administering medications such as benzodiazepines. After the administration, substance P and glutamate blood levels are measured to be lower than before. The patient optionally receives additional treatments such as non-pharmacologic treatment and active exercise. The patient also receives B-12 and/or folate vitamin supplements.

The description of ranges also describes the ranges within the specifically described range and describes individual numerical points for treatment. The described ranges are inclusive of endpoints in the range.

It should be understood that the present description of embodiments of the invention includes a composition for use in treating the conditions.

The usage of terms “may,” “can,” or similar terms herein are only for the purpose of flexibility in the disclosure and include, but are not limited to, the meaning of “is,” “are,” or similar terms.

It should be understood that the above description of embodiments of the invention and specific examples, while indicating preferred embodiments of the present invention, are given by way of illustration and not limitation. Many changes and modifications within the scope of the present invention may be made without departing from the spirit thereof, and the present invention includes all such changes and modifications.

What is claimed is:

1. A method of treating delirium in a patient in need thereof, the method comprising:
administering nitrous oxide and oxygen to the patient by inhalation, and
administering B-12 and/or folic acid to the patient, thereby treating delirium.

2. The method of claim 1, wherein a composition of the nitrous oxide and oxygen comprises from about 1% nitrous oxide/about 99% oxygen to about 70% nitrous oxide/about 30% oxygen.

3. The method of claim 2, wherein the composition of the nitrous oxide and oxygen comprises from about 40% nitrous oxide/about 60% oxygen to about 50% nitrous oxide/about 50% oxygen.

4. The method of claim 3, wherein the composition of the nitrous oxide and oxygen comprises about 50% nitrous oxide/about 50% oxygen.

5. The method of claim 1, wherein the nitrous oxide and oxygen are provided to an adult who weighs about 150 lbs. between about for about between 1 minute and about 1 hour every about 4-8 hours.

6. The method of claim 5, wherein the nitrous oxide and oxygen are provided to an adult who weighs about 150 lbs. between about for about 20 minutes every about 6-8 hours.

7. The substance of claim 5, wherein a composition, duration, an interval, and a total amount of the nitrous oxide and oxygen provided to an adult, or a child is adjusted for age, weight, or a combination thereof.

8. The method of claim 3, wherein the nitrous oxide and oxygen are provided to an adult who weighs about 150 lbs. between about for about between 1 minute and about 1 hour every about 4-8 hours.

9. The method of claim 8, wherein the nitrous oxide and oxygen are provided to an adult who weighs about 150 lbs. between about for about 20 minutes every about 6-8 hours.

10. The method of claim 1, wherein the nitrous oxide and oxygen are provided by continuous administration over the period of time.

11. The method of claim 1, further comprising monitoring if the patient's neuroexcitatory substances level normalizes.

12. The method of claim 2, wherein the neuroexcitatory substances comprise substance P or glutamate.

13. The method of claim 11, wherein the monitoring if the patient's neuroexcitatory substances level normalizes comprises monitoring if the patient's blood glutamate level normalizes.

14. The method of claim 11, wherein the patient's neuroexcitatory substances level is blood level.

15. The method of claim 14, wherein a normal blood glutamate level is from about 40 to about 60 uM.

16. The method of claim 11, wherein if the patient's neuroexcitatory substances level fails to normalize, additional nitrous oxide and oxygen are administered to the patient by inhalation.

17. The method of claim 11, further comprising monitoring the patient's physical symptoms.

18. The method of claim 17, wherein if the patient's physical symptom continues, additional nitrous oxide and oxygen is administered to the patient.

19. The method of claim 1, wherein both B-12 and folic acid are administered to the patient.

20. The method of claim 1, wherein B-12, if administered to the patient, is administered by mouth, and folic acid, if administered to the patient, is administered sublingual or by injection.
